(19) United States
(12) Patent Application Publication
(10) Pub. No.: US 2021/0128287 A1
(43) Pub. Date: May 6, 2021

(54) INCONTINENCE PREVENTION DEVICE

(71) Applicant: April Arafa, Greensboro, NC (US)

(72) Inventor: April Arafa, Greensboro, NC (US)

(21) Appl. No.: 16/669,085

(22) Filed: Oct. 30, 2019

(51) Int. Cl. A61F 2/00

(52) U.S. Cl. CPC A61F 2/005; A61F 2250/0078; A61F 2250/0008; A61F 2230/0071

(57) ABSTRACT

An incontinence prevention device to be inserted in a vagina, the incontinence prevention device including a bulbous member to contact at least a portion of a bladder to prevent a urinary leak from the bladder, a curved ridge circumferentially disposed around a bottom portion of the bulbous member to contact an interior surface of the vagina, such that the bulbous member is prevented from movement after insertion, and a base member disposed at a base of the bulbous member to facilitate extracting the bulbous member from the vagina.

### BACKGROUND

### 1. Field

The present general inventive concept relates generally to an incontinence device, and particularly, to an incontinence prevention device.

### 2. Description of the Related Art

Incontinence is a bladder control problem that results in urine leaking from the bladder involuntarily. The cause of incontinence can be due to an infection, food, stress, and/or a weakness in physical control.

Moreover, incontinence can affect men, women, and children of any age. Unfortunately, for many women, finding solutions to prevent incontinence can be difficult. Many products currently on the market for incontinence are outdated, bulky, and uncomfortable at best.

Therefore, there is a need for an incontinence prevention device that is portable, reusable, and applicable in a wide variety of situations.

### SUMMARY

The present general inventive concept provides an incontinence prevention device.

Additional features and utilities of the present general inventive concept will be set forth in part in the description which follows and, in part, will be obvious from the description, or may be learned by practice of the general inventive concept.

The foregoing and/or other features and utilities of the present general inventive concept may be achieved by providing an incontinence prevention device to be inserted in a vagina, the incontinence prevention device including a bulbous member to contact at least a portion of a bladder to prevent a urinary leak from the bladder, a curved ridge circumferentially disposed around a bottom portion of the bulbous member to contact an interior surface of the vagina, such that the bulbous member is prevented from movement after insertion, and a base member disposed at a base of the bulbous member to facilitate extracting the bulbous member from the vagina.

The bulbous member may deform in response to insertion within the vagina.

The curved ridge may include an adjustable section disposed within at least a portion of the curved ridge to facilitate changing the shape thereof.

The curved ridge may adjust to fit within the vagina by pivoting in at least one of a first direction and a second direction.

The base member may include a base surface, an elongated portion disposed on a first end at a center of the base surface to extend a predetermined distance away from the base surface, and a handle disposed on a second end of the elongated portion to facilitate gripping thereof.

### BRIEF DESCRIPTION OF THE DRAWINGS

These and/or other features and utilities of the present generally inventive concept will become apparent and more readily appreciated from the following description of the embodiments, taken in conjunction with the accompanying drawings of which:

FIG. 1 illustrates a side elevation view of an incontinence prevention device, according to an exemplary embodiment of the present general inventive concept.

### DETAILED DESCRIPTION

Various example embodiments (a.k.a., exemplary embodiments) will now be described more fully with reference to the accompanying drawings in which some example embodiments are illustrated. In the figures, the thicknesses of lines, layers and/or regions may be exaggerated for clarity.

Accordingly, while example embodiments are capable of various modifications and alternative forms, embodiments thereof are shown by way of example in the figures and will herein be described in detail. It should be understood, however, that there is no intent to limit example embodiments to the particular forms disclosed, but on the contrary, example embodiments are to cover all modifications, equivalents, and alternatives falling within the scope of the disclosure. Like numbers refer to like/similar elements throughout the detailed description.

It is understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.).

The terminology used herein is for the purpose of describing particular embodiments only and is not intended to be limiting of example embodiments. As used herein, the singular forms “a,” “an” and “the” are intended to include the plural forms as well, unless the context clearly indicates otherwise. It will be further understood that the terms “comprises,” “comprising,” “includes” and/or “including,” when used herein, specify the presence of stated features, integers, steps, operations, elements and/or components, but do not preclude the presence or addition of one or more other features, integers, steps, operations, elements, components and/or groups thereof.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art. However, should the present disclosure give a specific meaning to a term deviating from a meaning commonly understood by one of ordinary skill, this meaning is to be taken into account in the specific context this definition is given herein.

List of Components

Incontinence Prevention Device 100

Bulbous Member 110

Curved Ridge 120

Adjustable Section 121

Base Member 130

Base Surface 131

Elongated Portion 132

Handle 133

Aperture 133a

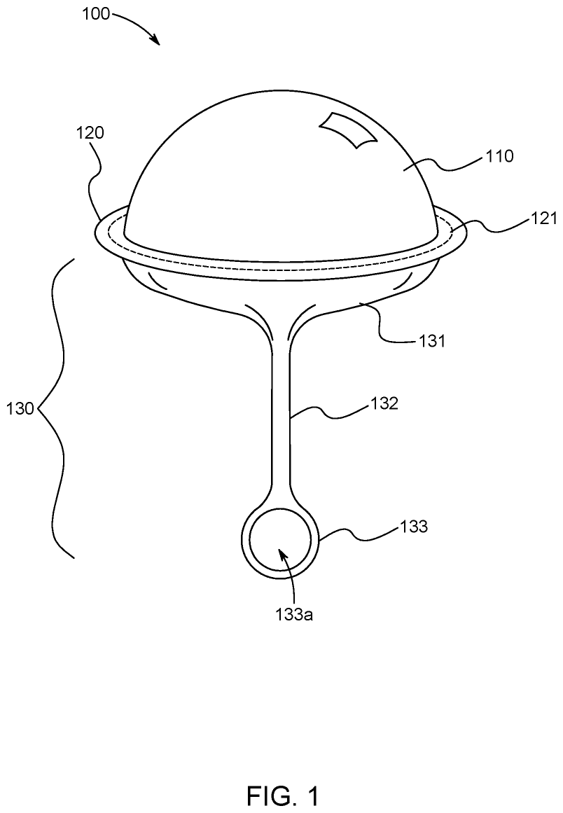

FIG. 1 illustrates a side elevation view of an incontinence prevention device 100, according to an exemplary embodiment of the present general inventive concept.

The incontinence prevention device 100 may be constructed from silicone and/or another type of medical grade rubber, etc., but is not limited thereto. The incontinence prevention device 100 may be highly durable and usable in various activities such as swimming and/or other exercise.

The incontinence prevention device 100 may include a bulbous member 110, a curved ridge 120, and a base member 130, but is not limited thereto.

Referring to FIG. 1, the bulbous member 110 is illustrated to have a semispherical shape. However, the bulbous member may be rectangular, conical, pentagonal, hexagonal, octagonal, or any other shape known to one of ordinary skill in the art, but is not limited thereto.

Moreover, the bulbous member 110 may deform in response to insertion in a vagina of a female. In other words, the bulbous member 110 may change shape to snugly fit within the vagina, such that the bulbous member 110 may become the shape of the vagina.

Furthermore, the bulbous member 110 may contact at least a portion of a bladder of the female to prevent a urinary leak from the bladder.

The curved ridge 120 may include an adjustable section 121, but is not limited thereto.

The curved ridge 120 may be circumferentially disposed around a bottom portion of the bulbous member 110 to contact an interior surface of the vagina, such that the bulbous member 110 may be prevented from movement after insertion therein. Additionally, the curved ridge 120 may pivot in a first direction or a second direction based on a preference of a user to adjust to fit within the vagina. Alternatively, the adjustable section 121 may deform in response to pressure applied by the user and/or after insertion in the vagina of the female. In other words, the curved ridge 120 may change shape to snugly fit within the vagina. Also, the adjustable section 121 may be disposed within at least a portion of the curved ridge 120 to facilitate changing the shape of the curved ridge 120.

The base member 130 may include a base surface 131, an elongated portion 132, and a handle 133, but is not limited thereto.

The base member 130 may be disposed at a base of the bulbous member 110. Additionally, the base member 130 may be disposed on at least a portion of a bottom of the curved ridge 120.

The base surface 131 may be constructed to be a round and/or curved surface.

The elongated portion 132 may be disposed on a first end at a center of the base surface 131 to extend a predetermined distance away from the base surface 131. Moreover, the elongated portion 132 may be a variable length based on a length of the vagina.

The handle 133 may include an aperture 133a, but is not limited thereto.

The handle 133 may be disposed on a second end of the elongated portion 132. The handle 133 may facilitate gripping thereof. Specifically, the aperture 133a may receive at least one finger therein to facilitate extracting the bulbous member 110 from the vagina.

The incontinence prevention device 100 may be cleaned and reused. Furthermore, the incontinence prevention device 100 may save on costs to prevent incontinence.

The present general inventive concept may include an incontinence prevention device 100 to be inserted in a vagina, the incontinence prevention device 100 including a bulbous member 110 to contact at least a portion of a bladder to prevent a urinary leak from the bladder, a curved ridge 120 circumferentially disposed around a bottom portion of the bulbous member 110 to contact an interior surface of the vagina, such that the bulbous member 110 is prevented from movement after insertion, and a base member 130 disposed at a base of the bulbous member 110 to facilitate extracting the bulbous member 110 from the vagina.

The bulbous member 110 may deform in response to insertion within the vagina.

The curved ridge 120 may include an adjustable section 121 disposed within at least a portion of the curved ridge 120 to facilitate changing the shape thereof.

The curved ridge 120 may adjust to fit within the vagina by pivoting in at least one of a first direction and a second direction.

The base member 130 may include a base surface 131, an elongated portion 132 disposed on a first end at a center of the base surface 131 to extend a predetermined distance away from the base surface 131, and a handle 133 disposed on a second end of the elongated portion 132 to facilitate gripping thereof.

Although a few embodiments of the present general inventive concept have been shown and described, it will be appreciated by those skilled in the art that changes may be made in these embodiments without departing from the principles and spirit of the general inventive concept, the scope of which is defined in the appended claims and their equivalents.

1. An incontinence prevention device to be inserted in a vagina, the incontinence prevention device comprising:
a bulbous member to contact at least a portion of a bladder to prevent a urinary leak from the bladder;
a curved ridge circumferentially disposed around a bottom portion of the bulbous member to contact an interior surface of the vagina, such that the bulbous member is prevented from movement after insertion; and
a base member disposed at a base of the bulbous member to facilitate extracting the bulbous member from the vagina.

2. The incontinence prevention device of claim 1, wherein the bulbous member deforms in response to insertion within the vagina.

3. The incontinence prevention device of claim 1, wherein the curved ridge comprises:
an adjustable section disposed within at least a portion of the curved ridge to facilitate changing the shape thereof.

4. The incontinence prevention device of claim 1, wherein the curved ridge adjusts to fit within the vagina by pivoting in at least one of a first direction and a second direction.

5. The incontinence prevention device of claim 1, wherein the base member comprises:
a base surface;
an elongated portion disposed on a first end at a center of the base surface to extend a predetermined distance away from the base surface; and
a handle disposed on a second end of the elongated portion to facilitate gripping thereof.
